(19) United States
(12) Patent Application Publication
(10) Pub. No.: US 2025/0025395 A1
(43) Pub. Date: Jan. 23, 2025

(54) Means for Oxidatively Dyeing Keratin Fibers

(71) Applicant: Henkel AG & Co. KGaA, Duesseldorf (DE)

(72) Inventors: Thomas Hippe, Appen (DE); Astrid Kleen, Haseldorf (DE); Hartmut Manneck, Barnitz (DE); Stefan Hoepfner, Hamburg (DE); Tugce Cansev, Hamburg (DE)

(21) Appl. No.: 18/890,263

(22) Filed: Sep. 19, 2024

Related U.S. Application Data
(63) Continuation of application No. PCT/EP2023/050061, filed on Jan. 3, 2023.

(30) Foreign Application Priority Data
Mar. 21, 2022 (DE) 102022202755.8

(51) Int. Cl. A61K 8/44; A61Q 5/10

(52) U.S. Cl. CPC A61K 8/44; A61Q 5/10; A61K 2800/20; A61K 2800/4324

(57) ABSTRACT

An agent for oxidatively dyeing keratinous fibers, in particular human hair, using at least one oxidation dye precursor of the developer type in combination with isatin and at least one determined complexing agent to provide a resorcinol-free hair dye.

### FIELD OF THE INVENTION

Agent for oxidatively dyeing keratin fibers, containing at least one oxidation dye precursor of the developer type, isatin, and at least one specific complexing agent

The present invention relates to cosmetic agents for oxidatively dyeing keratin fibers, which contain at least one oxidation dye precursor of developer type (a), isatin (b), and at least one complexing agent (c) from the group consisting of ethylenediamine disuccinic acid (EDDS), L-glutamic acid, N-diacetic acid (MGDA), N,N-bis(carboxymethyl)-L-alanine (MGDA), 1-hydroxy-1,1-diphosphonic acid (HEDP), ethylenediamine tetraacetic acid (EDTA), ethylenediamine tetramethyl phosphonic acid (EDTMP), diethylenetriamine pentamethylene phosphonic acid (DTPMP), pyridine-2,6-dicarboxylic acid, and the physiologically acceptable salts thereof.

A further subject matter is a multicomponent packaging unit (kit-of-parts) which, packaged separately in a first container, comprises an agent having the ingredients (a) and (b) and in a further container an oxidizing agent preparation containing hydrogen peroxide and (c).

A further object is a method for the oxidative dyeing of keratin fibers, in which a previously described agent is applied to the keratin fibers and rinsed out again after an exposure time.

### BACKGROUND OF THE INVENTION

In order to provide color-changing cosmetic agent, in particular for keratinous fibers, such as hair, a person skilled in the art is aware of various dyeing systems depending on the coloration requirement. So-called oxidative dyeing agents are used for permanent, intense coloring having appropriate fastness properties. Such dyeing agents typically contain oxidation dye precursors, known as developer components, and coupler components, which together form the actual dyes under the influence of oxidizing agents, for example hydrogen peroxide. Oxidation dyeing agents are characterized by outstanding, long-lasting coloring results.

The oxidation dye precursors (developers and couplers) themselves are not colored, but rather the actual dyeing agents are formed only during the course of the application when the oxidation dye precursors come into contact with the oxidizing agent (hydrogen peroxide). In a chemical reaction, the developers used as oxidation dye precursors (such as p-phenylenediamine or its derivatives) are first oxidatively converted by hydrogen peroxide into a reactive intermediate, also called quinonimine or quinone diimine, which then reacts in an oxidative coupling reaction with the couplers to form the corresponding dye.

With oxidation dyeing agents, hair can be dyed both in intensive fashion shades and in natural shades by choosing the suitable developer components and coupler components. A large area of application for oxidation dyes is the coloring of gray hair in a natural shade, which resembles the hair color that the user had when they were younger. A person skilled in the art knows the use of developers based on the base body of 1,4-diaminobenzene (para-phenylenediamine) and couplers with a resorcinol structure (1,3-dihydroxybenzene) as a classic combination for producing oxidative colorations in the brown to dark-blond range.

Said oxidation dyes have been used for decades. Although they are only intended for extracorporeal use on keratin fibers such as headhair, eyelashes, and eyebrows, contact of the dye with the scalp cannot be completely avoided during use. In order to ensure the highest possible product safety for customers, the commercially available oxidation dye precursors are continuously checked for their physiological compatibility, for example by the Scientific Committee on Consumer Products (SCCP), an advisory body of the European Commission. It is known that some of the oxidation dye precursors, in particular some of the oxidation bases of the para-phenylenediamine type, can have a certain potential for sensitization. In order to rule out allergic reactions during or after the dyeing process, the customer is therefore recommended to perform a test with a small amount of the dye on the skin before using the dye on the hair. In addition to skin sensitization, other physiological effects are also monitored.

Resorcinol, 4-chlororesorcinol and 2-methylresorcinol are common oxidation dye precursors with a 1,3-dihydroxybenzene base body. In its last opinion from March 2021, SCCP came to the conclusion that the use of resorcinol in oxidative hair dyes with a resorcinol concentration of up to 1.25 percent by weight in the ready-to-use mixture was considered safe. The SCCP stated that resorcinol has a thyroid-inhibiting effect. Although a definite level of exposure required for such an effect cannot be derived from the available studies in humans, most of these studies indicate a much higher exposure relatively than is the case in cosmetics.

### BRIEF SUMMARY OF THE INVENTION

In order to take into account the concerns of some consumers with regard to product safety, the object of the present invention was to provide an agent for oxidatively dyeing keratin fibers, in particular human hair, with which a broad color spectrum can be covered, in particular a natural color pallet with cool natural shades and warm natural shades, and a gold color series which leads to colors having high fastness properties, without impairing product safety. In particular, dyeing in these natural shades should be possible without the use of couplers of the recycling type.

Many users dye their hair over decades in the same shade and do not want a sudden, obvious, visible change to their usual hair color. For these users, it is therefore essential to enable the customary, resorcinol-containing hair dye to be replaced by a new, resorcinol-free product without any change in shade. A central challenge of the present application was therefore to find a new, resorcinol-free hair dye which, in its color effect and its color result, corresponds as precisely as possible to the resorcinol-containing dyeing agent used to date.

Surprisingly, it could now be found that this object can be achieved excellently by an oxidative dyeing agent which contains an oxidation dye precursor of developer type (a), isatin (b), and at least one complexing agent (c) from the group consisting of ethylenediamine disuccinic acid (EDDS), L-glutamic acid, N-diacetic acid (MGDA), N,N-bis(carboxymethyl)-L-alanine (MGDA), 1-hydroxy-ethane-1,1-diphosphonic acid (HEDP), ethylenediamine tetraacetic acid (EDTA), ethylenediamine tetramethylene phosphonic acid (EDTMP), diethylenetriamine pentamethylene phosphonic acid (DTPMP), pyridine-2,6-dicarboxylic acid, and the physiologically acceptable salts thereof.

### DETAILED DESCRIPTION OF THE INVENTION

A first subject matter of the present invention is an agent for oxidatively dyeing keratinous fibers, in particular human hair, containing, in a cosmetic carrier,
(a) at least one developer-type oxidation dye precursor, and
(b) isatin and
(c) at least one complexing agent from the group consisting of ethylenediamine disuccinic acid (EDDS), L-glutamic acid-N,N-diacetic acid (GLDA), N,N-bis(carboxymethyl)-L-alanine (MGDA), 1-hydroxyethane-1,1-diphosphonic acid (HEDP), ethylenediaminetetraacetic acid (EDTA), ethylenediamine tetramethylene phosphonic acid (EDTMP), diethylene triamine pentamethylene phosphonic acid (DTPMP), pyridine-2,6-dicarboxylic acid, and the physiologically acceptable salts thereof.

The work leading to this invention has shown that the oxidative dyeing of hair using a developer (a) in combination with isatin (a) leads to very intense dyeing with excellent fastness properties if a complexing agent (c) from the above-mentioned specific group (b) is still used in the dyeing agent.

### Keratin Fibers

Keratin fibers are, in principle, understood to mean all types of animal hair, for example wool, horsehair, angora hair, furs, feathers, and products or textiles manufactured therefrom. Preferably, however, the keratin fibers are human hair.

### Agent for Oxidation Dyeing

The term “agent for oxidation dyeing” of keratin fibers used according to the invention is understood to mean oxidation dyes. Oxidative dyeing agents contain oxidation dye precursors, so-called developers and coupler components. Developers and couplers diffuse separately into the keratin fibers and, in a chemical reaction with one another, form the actual dyes under the influence of an alkalizing agent (e.g., ammonia) and an oxidizing agent (hydrogen peroxide). Depending on the quantity of oxidizing agent used, the keratin fibers are simultaneously lightened to a greater or less extent during coloring, since the oxidizing agent not only initiates the dye-forming process of the developers and couplers, but also oxidatively destroys the hair's own pigments (melanins). Depending on the used amounts of the oxidation dye precursor products and of the oxidizing agent, the oxidative coloration can therefore be predominantly a coloration (with high dye proportion) or a lightening (with high proportion of oxidizing agent). In the latter case, the oxidation dye precursors are mostly used to tone the brightening result.

The agent according to the invention contain the components essential to the invention in each case in a cosmetic carrier, preferably in a suitable aqueous, alcoholic, or aqueous-alcoholic carrier. For the purpose of hair coloring, such carriers are, for example, creams, emulsions, gels, or also surfactant-containing foaming solutions, such as shampoos, foam aerosols, foam formulations, or other ingredients which are suitable for use on hair.

The oxidative dye described above is a ready-to-use dye which is applied in this form containing the components (a) and (b) and (c) for application onto the keratin fibers.

### Oxidation Dye Precursors of Developer Type (a)

Oxidative dyeing agents contain oxidation dye precursors, so-called developers and coupler components, for the formation of the coloring. Developers and couplers diffuse separately into the keratin fibers and form the actual dyes in a chemical reaction with one another under the influence of ammonia as alkalizing agent and an oxidizing agent. Depending on the quantity of oxidizing agent used, the keratin fibers are simultaneously lightened to a greater or less extent during coloring, since the oxidizing agent not only initiates the dye-forming process of the developers and couplers, but also oxidatively destroys the hair's own pigments (melanins). Depending on the used amounts of the oxidation dye precursor products and of the oxidizing agent, the oxidative color change can therefore be predominantly a coloration (with high dye proportion) or a lightening (with high proportion of oxidizing agent). In the latter case, the oxidation dye precursors are mostly used to tone the brightening result.

As a first substantial component, the oxidative dyeing agents according to the invention contain at least one oxidation dye precursor of the developer type, also referred to as developer for short.

At least one oxidation dye precursor of the developer type, selected from the group including p-toluenediamine, 2-methoxymethyl-p-phenylenediamine, 2-(2-hydroxy-hydroxyethyl)-p-phenylenediamine, N,N-bis(2-hydroxy-hydroxyethyl)-p-phenylenediamine, p-phenylenediamine and the physiologically acceptable salts thereof.

In a particularly preferred embodiment, an agent according to the invention is therefore characterized in that it contains at least one oxidation dye precursor of the developer type (a) which is selected from the group of p-toluylenediamine, 2-methoxymethyl-p-phenylenediamine, (2-hydroxyethyl)-p-phenylenediamine, N,N-bis-(2-hydroxyethyl)-p-phenylenediamine, p-phenylenediamine, and the physiologically acceptable salts thereof. p-toluylenediamine is alternatively also referred to as 2,5-toluylenediamine, p-toluylenediamine (abbreviation: PTD), 2,5-diaminotoluene, 2-methyl-p-phenylenediamine or 2,5-diaminomethylbenzene. PTD has the CAS number 95-70-5. 2-methoxymethyl-p-phenylenediamine is alternatively also referred to as 2-methoxymethyl-1,4-benzenediamine and, in the form of its free base, bears the CAS number 337906-36-2. 2-(2-hydroxyethyl)-p-phenylenediamine is alternatively referred to as 2-(2,5-diaminophenyl) ethanol and, in the form of its free base, carries the CAS number 93841-24-8. N,N-bis-(2-hydroxyethyl)-p-phenylenediamine, in the form of its free base, has the CAS number 7575-35-1.

With an oxidative dyeing agent, which contains a developer (a) of the aforementioned group with the base structure of the 1,4-diaminobenzene, hair was able to be colored with very high intensity in natural shades, in particular in dark brown, medium brown and dark blonde shades. It was also particularly surprising that the hair could be colored in a shade which was very particularly similar to the shade obtained with a dye containing the classic combination of PDT and resorcinol.

The agent which contains at least one oxidation dye precursor of the developer type (a), which is selected from the group of p-toluylenediamine, 2-methoxymethyl-p-phenylenediamine and the physiologically acceptable salts thereof, has proven to be very particularly suitable for achieving the object according to the invention.

In a particularly preferred embodiment, an agent according to the invention is therefore characterized in that it contains at least one oxidation dye precursor of the developer type (a) which is selected from the group of p-toluylenediamine, 2-methoxymethyl-p-phenylenediamine and the physiologically acceptable salts thereof.

However, the agent according to the invention can also contain other developers (a) to form shades with a natural reddish tone. For example, developers from the group of 4-amino-3-methylphenol, p-chlorophenol, 4,5-diamino-1-(2-hydroxy-hydroxyethyl)-pyrazole and their physiologically acceptable salts are particularly suitable for this purpose. These developers can be used either as a group on their own or also together with one or more developers having a basic structure of p-phenylenediamine.

In a further preferred embodiment, an agent according to the invention is therefore characterized in that it contains at least one oxidation dye precursor of developer type (a) which is selected from the group consisting of 4-amino-3-methylphenol, p-aminophenol, 4,5-diamino-1-(2-hydroxyethyl)-pyrazole and the physiologically acceptable salts thereof.

In a further particularly preferred embodiment, an agent according to the invention is therefore characterized in that it comprises at least one oxidation dye precursor of the developer type (a), which is selected from the group consisting of p-toluylene-diamine, 2-methoxymethyl-p-phenylenediamine, 2-(2-hydroxy-ethyl)-p-phenylenediamine, N,N-bis-(2-hydroxy-ethyl)-p-phenylenediamine, p-phenylenediamine, 4-amino-3-methyl-phenol, p-amino-phenol, 4,5-diamino-1-(2-hydroxy-ethyl)-pyrazole and physiologically tolerated salts thereof.

The developers of the above-mentioned group (a) can be used in the agent according to the invention in the form of their free base or else in the form of their physiologically acceptable salts. A physiologically acceptable salt is understood to mean a salt of the developer that is well tolerated by the user under physiological conditions, i.e., during use of the agent. Physiologically acceptable salts are in particular the chlorides, bromides, sulfates, and hemisulfates of developers (a).

In a further particularly preferred embodiment, an agent according to the invention is therefore characterized in that it contains at least one oxidation dye precursor of developer type (a) which is selected from the group of p-toluylenediamine, p-toluylenediamine sulfate, p-toluylenediamine chloride, p-toluylenediamine bromide, 2-methoxymethyl-p-phenylenediamine, 2-methoxymethyl-p-phenylenediamine sulfate, 2-methoxymethyl-p-phenylenediamine chloride and 2-methoxymethyl-p phenyldiamine bromide, 4,5-diamino-1-(2-hydroxyethyl)-pyrazole chloride, 4,5-diamino-1-(2-hydroxyethyl)-pyrazole bromide and 4,5-diamino-1-(2-hydroxyethyl) pyrazole sulfate.

Depending on the desired color effect, it can furthermore be preferred for the agent to additionally contain one or more further oxidation dye precursors of the developer type, which are selected from the group of bis-(2-hydroxy-5-aminophenyl) methane, 1,3-bis-(2,5-diaminophenoxy) propan-2-ol, N, N′-bis-(4-aminophenyl)-1,4-diazacycloheptane, 1,10-bis-(2,5-diaminophenyl)-1,4,7,10-tetraoxadecane, p-aminophenol, 4-amino-2-methylphenol, 4-amino-2-aminomethylphenol, 4-amino-2-(1,2-dihydroxyethyl) phenol, 4-amino-2-(diethylaminomethyl) phenol, 4,5-diamino-1-(2-hydroxyethyl) pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H, 5H-pyrazolo[1,2-a]pyrazol-1-one, and the physiologically acceptable salts thereof.

The developer(s) is/are preferably used in specific quantity ranges in the agent according to the invention. The agent preferably contains, based on the total weight of the agent, one or more oxidation dye precursors of developer type (a) in a total amount of from 0.001 to 10.0 wt. %, preferably from 0.01 to 6.0 wt. %, more preferably from 0.1 to 5.0 wt. %, and very particularly preferably from 0.15 to 4.7 wt. %.

In a particularly preferred embodiment, a agent according to the invention is thus characterized in that the agent contains, based on the total weight of the agent, one or more oxidation dye precursors of developer type (a) in a total amount of 0.001 to 10.0 wt. %, preferably 0.01 to 6.0 wt. %, more preferably 0.1 to 5.0 wt. %, and very particularly preferably 0.15 to 4.7 wt. %.

### Isatin

As a second component substantial to the invention, the oxidation dye is isatin (b). Isatin is the compound of formula (ISA), which can alternatively also be referred to as 2,3-indoline-dione or as 2,3-dioxoindoline

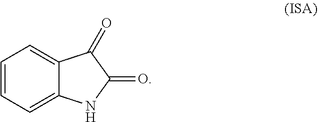 [SMILES: O=C1Nc2ccccc2C1=O]

Isatin has the CAS number 91-56-5.

With regard to an optimal solution of the object according to the invention, isatin (b) is preferably contained in specific quantity ranges in the agent according to the invention. Particularly good results were obtained when the agent contained, based on the total weight of the agent, 0.001 to 10 wt. %, preferably 0.01 to 5 wt. %, more preferably 0.1 to 3.5 wt. %, and very particularly preferably 0.15 to 2.5 wt. % isatin (b).

In a particularly preferred embodiment, a agent according to the invention is thus characterized in that it contains, based on the total weight of the agent, 0.001 to 10 wt. %, preferably 0.01 to 5 wt. %, more preferably 0.1 to 3.5 wt. %, and particularly preferably 0.15 to 2.5 wt. % isatin (b).

Isatin is commercially available from different suppliers, such as Acros, Sigma Aldrich, Thermo Scientific, etc.

### Weight Ratio of Developers (a) to Isatin (b) on Average

The shade resulting from the coloration on the hair depends both on the amounts of the developer(s) (a) used and on the amount of the isatins (b) contained in the agent. As is known from typical dyeing practice, the higher the amounts of developers (a) and isatin (b) used, the higher the intensity of the coloration. However, the nature of the resulting coloring can be controlled by the quantitative ratio in which the developers of group (a) and isatin (b) are used in the oxidative dye.

The colors produced on the hair then had a particularly high similarity to the colorations obtained with the couplers of the resorcinol type if the agent contained the developers (a) and isatin (b) in a weight ratio which has value of from 2:1 to 1:2, preferably from 1.9:1 to 1:1, more preferably from 1.9:1 to 1.1:1, even more preferably from 1.8:1 to 1.1:1, and very particularly preferably from 1.7:1 to 1.2:1. This effect was observed in particular in the darker natural shades, such as dark brown and medium blond.

In a particularly preferred embodiment, a agent according to the invention is thus characterized in that the weight ratio of all the developers of the group (a) contained in the agent to the isatin (b) contained in the agent, i.e., the weight ratio (a)/(b), has a value of 2:1 to 1:2, preferably of 1.9:1 to 1:1, more preferably of 1.9:1 to 1.1:1, even more preferably of 1.8:1 to 1.1:1 and very particularly preferably of 1.7:1 to 1.2:1.

In the preferred weight ratio (a)/(b) of 1.9:1 to 1:1, for example, the developer (a) or developers (a) is/are used in the agent either in the same amount as isatin (b) or in an up to 1.9-fold weight excess. Very particularly preferably, in comparison with the isatin (b), the developers (a) are used in a 1.2-fold to 1.7-fold weight excess.

### Further Couplers (D) in Oxidation Dyes

For precise shading and/or fine adjustment of the desired color shade, the oxidative dye can also contain further couplers (d) in addition to the oxidation dye precursor(s) of the developer type (a) and isatin (b).

Further very well suited couplers can be selected for example from the group consisting of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy) ethanol, 1,3-bis(2,4-diaminophenoxy) propane, 1-methoxy-2-amino-4-(2-hydroxyethylamino)benzene, 1,3-bis(2,4-diaminophenyl) propane, 2,6-bis(2′-hydroxyethyl)-1-methylbenzene, 1-amino-3-bis(2-hydroxyethyl)aminobenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

In a particularly preferred embodiment, an agent according to the invention is thus characterized in that it contains at least one oxidation dye precursor of the coupler type (d) which is selected from the group of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2 (2,4-diaminophenoxy) ethanol, 1,3-bis(2,4-diaminophenoxy) propane, 1-methoxy-2-amino-4-(2-hydroxyethylamino)benzene, 1,3-bis(2,4-diaminophenyl) propane, 2,6-bis(2′-hydroxyethyl)-1-methylbenzene, 1-amino-3-bis(2-hydroxyethyl)aminobenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

Particularly natural shades with great similarity to the corresponding resorcinol-containing dyeing agents could be obtained if the dye additionally contained one or more couplers (d) which were selected from the group consisting of 3-aminophenol, 5-amino-2-methylphenol, 3-amino-2-chloro-6-methylphenol, 2-amino-3-hydroxypyridine, 1-methoxy-2-amino-4-(2-hydroxyethylamino)benzene, 6-hydroxyindole, or mixtures of these compounds or the physiologically acceptable salts thereof. For this reason, the use of couplers (d) from this group is explicitly very particularly preferred.

In an explicitly very particularly preferred embodiment, an agent according to the invention is therefore characterized in that it contains at least one oxidation dye precursor of coupler type (d) which is selected from the group consisting of 3-aminophenol, 5-amino-2-methylphenol, 3-amino-2-chloro-6-methylphenol, 2-amino-3-hydroxypyridine, 1-methoxy-2-amino-4-(2-hydroxyethyl amino)benzene, 6-hydroxyindole, or mixtures of said compounds or the physiologically acceptable salts thereof.

The couplers from the above-described group (d) are also preferably used in specific quantity ranges in the agent according to the invention. Particularly positive results were obtained when the agent contained, based on the total weight of the agent, one or more oxidation dye precursors of the coupler type (d) in a total amount of 0.001 to 10 wt. %, preferably 0.01 to 5 wt. %, more preferably 0.1 to 3.5 wt. %, and very particularly preferably 0.15 to 2.5 wt. %.

In a further preferred embodiment, a agent according to the invention is characterized in that the agent contains, based on the total weight of the agent, one or more oxidation dye precursors of the coupler type (d) in a total amount of 0.001 to 10 wt. %, preferably 0.01 to 5 wt. %, more preferably 0.1 to 3.5 wt. %, and very particularly preferably 0.15 to 2.5 wt. %.

Dispensing with Resorcinol-Type Couplers

As already described above, with the agents of the present application, intense colorations in the natural tone range should be developed, which reproduce the shades created with resorcinol-containing

Dyeing agents as well as possible without being dependent on the use of couplers of the resorcinol type.

Resorcinol-type couplers or couplers from the group of resorcinols are understood to mean 1,3-dihydroxybenzene and its derivatives. Derivatives of 1,3-dihydroxybenzene are all compounds which have a 1,3-dihydroxybenzene basic structure and carry further substituents, but both hydroxyl groups of the 1,3-dihydroxybenzene must still be present.

The couplers from the group of resorcinols used as standard in market products are resorcinol, 2-methylresorcinol, and 4-chlororesorcinol. Couplers from the group of resorcinols are therefore understood in particular to be resorcinol, 2-methylresorcinol, and 4-chlororesorcinol. In the agent of the present application, these couplers are to be dispensed with, and therefore it is preferred if the total amount of precursors of the coupler type contained in the agent from the group of resorcinols, in particular from the group consisting of resorcinol, 2-methylresorcinol, and 4-chlororesorcinol, is below 0.1 wt. %, preferably below 0.05 wt. %, particularly preferably below 0.01 wt. %, and very particularly preferably 0 wt. %.

In a further very particularly preferred embodiment, a agent according to the invention is therefore characterized in that, based on the total weight of the agent, the total amount of the oxidation dye precursors of the coupler type contained in the agent is from the group of resorcinols, in particular from the group consisting of resorcinol, 2-methylresorcinol, and 4-chlororesorcinol, is below 0.1 wt. %, preferably below 0.05 wt. %, particularly preferably below 0.01 wt. %, and very particularly preferably 0 wt. %.

### Substantive Dyes

Furthermore, the agents according to the invention can optionally contain at least one substantive dye. These are dyeing agents which are drawn directly onto the hair and which do not require an oxidative process in order to form the color, substantive agents are typically nitrophenylenediamines, nitroaminophenols, azo dyeing agents, anthraquinones, triarylmethane dyeing agents, or indophenols.

The substantive dyeing agents are each used preferably in an amount of from 0.001 to 20 wt. %, in particular from 0.05 to 5 wt. %, in each case in relation to the total preparation for use. The total quantity of substantive dyeing agents is preferably at most 3 wt. %.

Substantive dyes can be divided into anionic, cationic and non-ionic substantive dyes, which are selected and used by a person skilled in the art according to the requirements of the support base.

Preferred anionic substantive agents are the compounds known under the international names or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, and Acid Black 52.

Preferred cationic direct dyes are Basic Blue 7, Basic Blue 26, Basic Violet 2 und Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Yellow 87, Basic Orange 31, and Basic Red 51.

In particular, non-ionic nitro dyes and quinone dyes and neutral azo dyes are suitable as non-ionic direct dyes. Suitable non-ionic substantive agents are the compounds known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid, and 2-chloro-6-ethylamino-4-nitrophenol.

### Hydrogen Peroxide (E)

For the formation of the colors in the oxidation dyeing process, the oxidation dyeing agent according to the invention preferably contains at least one oxidizing agent (e) in which are particularly preferably hydrogen peroxide and/or the addition products of hydrogen peroxide of organic or inorganic compounds.

In a preferred embodiment, hydrogen peroxide itself is used as an aqueous solution in the oxidation dye. The concentration of a hydrogen peroxide solution is determined by the legal requirements, on one hand, and by the desired effect, on the other; preferably, 6 to 12 wt. % solutions in water are used. Oxidative that are preferred according to the invention are characterized in that they contain 0.5 to 20 wt. %, preferably 1 to 12.5 wt. %, especially preferably 2.5 to 10 wt. %, and, in particular, 3 to 8 wt. % hydrogen peroxide, based in each case on the total weight of the oxidative dye.

In a further particularly preferred embodiment, an agent according to the invention is therefore characterized in that it contains at least one oxidizing agent (e) from the group consisting of hydrogen peroxide and its addition products of organic or inorganic compounds.

In a particularly preferred embodiment, an agent according to the present invention is characterized in that it contains, based on the total weight of the agent, 0.5 to 20 wt. %, preferably 1 to 12.5 wt. %, more preferably 2.5 to 10 wt. %, and particularly preferably 3 to 8 wt. % hydrogen peroxide (e).

### Complexing Agents (C)

As a third component essential to the invention, the oxidative dyeing agents according to the invention contain at least one complexing agent (c) from the group consisting of ethylenediamine disuccinic acid (EDDS), L-glutamic acid-N, N-diacetic acid (GLDA), N,N-bis(carboxymethyl)-L-alanine (MGDA), 1-hydroxy-ethane-1,1-diphosphonic acid (HEDP), ethylenediaminetetraacetic acid (EDTA), ethylenediamine tetramethylene phosphonic acid (EDTMP), diethylenetriamine-pentamethylenephosphonic acid (DTPMP), pyridine-2,6-dicarboxylic acid, and the physiologically acceptable salts thereof.

Ethylenediamine disuccinic acid or the salts thereof are often also abbreviated in the literature as EDDS. EDDS can be used in the Form of its free acid or else as a monosodium salt, as disodium salt, as trisodium salt or as tetrasodium salt. The corresponding potassium salts can also be used and are particularly preferred.

The trisodium salt of ethylenediamine disuccinate is alternatively also referred to in its (S, S) form as ethylene diamine N, N′disuccinic acid trisodium sodium salt * (S, S), carries the CAS number 178949-82-1, and can be commercially obtained, for example, under the trade name Nitraquest E 30 from Innospec Limited. Another alternative name for the trisodium salt of ethylenediamine disuccinate is N, N′-1,2-ethanediylbis-L-aspartic acid, trisodium salt. The substance can also be obtained under the trade names Enviomet C 140 and Octaquest E 30 from Innospec.

The free acid of this compound is referred to as ethylenediamine disuccinic acid. Ethylenediamine disuccinic acid has the CAS number 20846-91-7 and can alternatively also be referred to as N,N-1,2-ethanediylbis-L-aspartic acid or as ethylenediamine disuccinic acid.

L-glutamic acid-N, N-diacetic acid is abbreviated in the literature as GLDA and has the CAS number 51981-21-6. GLDA has the formula (KOM-I).

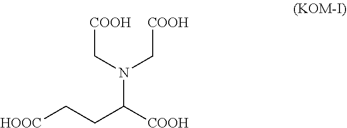 [SMILES: O=C(O)CCC(C(=O)O)N(CC(=O)O)CC(=O)O]

The salts of GLDA, for example the monosodium salt, the disodium salt and the tetrasodium salt are likewise according to the invention. The tetrasodium salt of the L-glutamic acid-N,N-diacetic acid is referred to as Tetranatrium-N,N-bis(carboxylatomethyl)-L-glutamate (GLDA-Na₄).

N, N-bis(carboxymethyl)-L-alanine (MGDA) is alternatively also referred to as 2-methyl-2′,2″,2″′-nitrilotriacetic acid and is abbreviated as the substance MGDA. MGDA has the CAS number 29578-05-0 and is commercially available from different suppliers, for example from ABClabtory Scientific Co. Ltd, from Chemieliva Pharmaceutical Co. Ltd., from SIA “Chemspace” or from Hong Kong Chemhere Co. Ltd,. MGDA has the formula (KOM II). The physiologically acceptable salts, for example the sodium salts, the potassium salts, or the ammonium salts of MGDA, are also according to the invention.

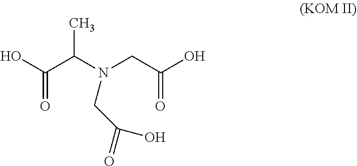

1-hydroxy-ethane-1,1-diphosphonic acid (HEDP), ethylenediaminetetraacetic acid (EDTA), ethylene diamine tetramethylene phosphonic acid (EDTMP), diethylenetriamine pentamethylene phosphonic acid (DTPMP), pyridine-2,6-dicarboxylic acid and the physiologically acceptable salts thereof are physiologically acceptable complexing agents.

The agents containing ethylenediamine disuccinic acid (EDDS), L-glutamic acid-N, N-diacetic acid (GLDA), N, N-bis(carboxymethyl)-L-alanine (MGDA) and/or physiologically acceptable salts thereof showed particularly good color results. For this reason, oxidative dyeing agents with these complexing agents (c) are very particularly preferred.

In a particularly preferred embodiment, an agent according to the invention is characterized in that it contains at least one complexing agent (c) from the group consisting of ethylenediamine disuccinic acid (EDDS), L-glutamic acid-N, N-diacetic acid (GLDA), N, N-Bis(carboxymethyl)-L-alanine (MGDA) and physiologically acceptable salts thereof.

In the alkali pH values of the treatment solutions required according to the invention, these complexing agents are present at least partially as anions. It does not matter whether they are introduced in the form of acids or in the form of salts. In the case of use as salts, alkali, ammonium or alkylammonium salts, in particular sodium salts, are preferred.

The complexing agents from the above-described group (c) are also preferably used in specific quantity ranges in the agent according to the invention.

For example, the dyeing agents can contain one or more complexing agents in a total amount of from 0.05 to 10.0 wt. %, preferably from 0.1 to 5.0 wt. %, more preferably from 0.15 to 2.5 wt. %, and very particularly preferably from 0.2 to 1.5 wt. %, wherein the figures in wt. % are each based on the total weight of the agent.

In a particularly preferred embodiment, an agent according to the invention is characterized in that the agent contains, based on the total weight of the agent, one or more complexing agents (c) in a total amount of 0.05 to 10.0 wt. %, preferably 0.1 to 5.0 wt. %, more preferably 0.15 to 2.5 wt. %, and very particularly preferably 0.2 to 1.5 wt. %.

### Alkalizing Agent

Coloring processes on keratin fibers typically take place in an alkaline environment. To protect the keratin fibers as well as the skin as much as possible, however, it is not desirable to adjust to too high a pH value. It is therefore preferred if the pH of the ready-to-use agent is between 6 and 11, in particular between 7 and 10.5. The pH values within the meaning of the present invention are pH values which have been measured at a temperature of 22° C.

The work leading to this invention has shown that good color results were obtained in particular if an alkalizing agent from the group consisting of ammonia, monoethanolamine, 2-amino-2-methylpropanol, arginine, lysine, ornithine, and histidine was used in the dyeing agent according to the invention. For this reason, it is also preferred if the dyeing agent according to the invention contains at least one alkalizing agent from the group consisting of ammonia, monoethanolamine, 2-amino-2-methylpropanol, arginine, lysine, ornithine, and histidine.

In a particularly preferred embodiment, an agent according to the invention is characterized in that it contains at least one alkalizing agent from the group consisting of ammonia, monoethanolamine, 2-amino-2-methylpropanol, arginine, lysine, ornithine, and histidine.

By using the suitable or preferred alkalizing agent, the pH value suitable for the oxidation dyeing process can be configured in the dyeing agent according to the invention, which is in the range of 6.5 to 11.5, preferably from 8.5 to 11.0, and very particularly preferably from 9.0 to 10.5.

In the context of a further preferred embodiment, an agent according to the invention is characterized in that it contains water and has a pH in the range of 6.5 to 11.5, preferably of 8.5 to 11.0, and very particularly preferably of 9.0 to 10.5.

For precise adjustment of the pH, the dyeing agent can also contain one or more acidifying agents in addition to the alkalizing agents. According to the invention, preferred acidifying agents are edible acids, such as citric acid, acetic acid, malic acid or tartaric acid, as well as dilute mineral acids. The pH values within the meaning of the present invention are pH values which have been measured at a temperature of 22° C.

### Additional Ingredients in the Agent

Preferably, an emulsifier or a surfactant is also added to the oxidative dyeing agents, wherein surface-active substances are referred to as surfactants or emulsifiers depending on the field of application and are selected from anionic, cationic, zwitterionic, amphoteric and nonionic surfactants and emulsifiers.

In a further very particularly preferred embodiment, an agent according to the invention is therefore characterized in that it contains at least one surfactant selected from the group consisting of anionic, amphoteric, zwitterionic, and non-ionic surfactants.

Suitable anionic surfactants in agents according to the invention are all anionic surface-active substances suitable for use on the human body. These are characterized by a water-solubilizing, anionic group, such as for example a carboxylate, sulfate, sulfonate or phosphate group, and a lipophilic alkyl group having about 8 to 30 carbon atoms. In addition, glycol ether or polyglycol ether groups, ester, ether and amide groups, and hydroxyl groups can be contained in the molecule. Examples of suitable anionic surfactants are, in each case in the form of the sodium, potassium and ammonium and of the mono-, di- and trialkanolammonium salts having 2 to 4 C atoms in the alkanol group,
linear and branched fatty acids having 8 to 30 C atoms (soaps);
ether carboxylic acids of the formula RO(CH₂CH₂O)ₓCH₂COOH, in which R is a linear alkyl group having 8 to 30 C atoms and x=0 or 1 to 16,
acyl sarcosides having 8 to 24 C atoms in the acyl group;
acyl taurides having 8 to 24 C atoms in the acyl group;
acyl isethionates having 8 to 24 C atoms in the acyl group;
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups;
linear alkane sulfonates having 8 to 24 C atoms;
linear α-olefin sulfonates having 8 to 24 C atoms;
sulfonates of unsaturated fatty acids having 8 to 24 C atoms and 1 to 6 double bonds,
αsulfo fatty acid methyl esters of fatty acids having 8 to 30 C atoms;
alkyl sulfates and alkyl ether sulfates of the formula RO(CH₂CH₂O)ₓSO₃H, in which R is a preferably linear alkyl group having 8 to 30 C atoms and x=0 or 1 to 12;
mixtures of surface-active hydroxy sulfonates,
sulfated hydroxyalkyl polyethylene and/or hydroxyalkylene propylene glycol ether,
esters of tartaric acid and citric acid with alcohols, which represent addition products of about 2-15 molecules ethylene oxide and/or propylene oxide to fatty alcohols having 8 to 22 C atoms,
alkyl and/or alkenyl ether phosphates of the formula

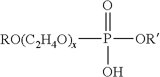

in which R preferably represents an aliphatic, optionally unsaturated hydrocarbon radical having 8 to 30 carbon atoms, R′ represents hydrogen, a radical (CH₂CH₂O)_{y}R and x and y independently of one another represent a number from 1 to 10,
sulfated fatty acid alkylene glycol esters of the formula RC(O)O(alkO)ₙSO₃, in which R represents a linear or branched, aliphatic, saturated and/or unsaturated alkyl functional group having 6 to 22 C atoms, alk represents CH₂CH₂, CHCH₃CH₂ and/or CH₂CH CH₃ and n represents a number from 0.5 to 5,
monoglyceride sulfates and monoglyceride ether sulfates.

Preferred anionic surfactants are alkyl sulfates, alkyl polyglycol ether sulfates, and ether carboxylic acids having 10 to 18 C atoms in the alkyl group and up to 12 glycol ether groups in the molecule.

Surface-active compounds that carry, in the molecule, at least one quaternary ammonium group and at least one carboxylate, sulfonate or sulfate groups are referred to as zwitterionic surfactants. Particularly suitable zwitterionic surfactants are the so-called betaines, such as the N-alkyl-N, N-dimethylammonium glycinates, for example the coconut alkyl dimethyl ammonium glycinate, N-acyl-aminopropyl-N,N-dimethylammonium glycinates, for example the coco-acylaminopropyldimethylammonium glycinate, and 2-alkyl-3-carboxymethyl-3-hydroxyethyl imidazolines, each having 8 to 18 C atoms in the alkyl or acyl group, and also the coco-acyl aminoethyl hydroxyethyl carboxymethyl glycinate. A preferred zwitterionic surfactant is the fatty acid amide derivative known by the INCI name cocamidopropyl betaine.

Amphoteric surfactants are understood to be surface-active compounds which, in addition to a C₈-C₂₄ alkyl or acyl group, also contain at least one free amino group and at least one —COOH— or —SO₃H group in the molecule and are capable of forming inner salts. Examples of suitable amphoteric surfactants are N-alkylglycines, N-alkylpropionic acids, N-alkylamino-butyric acids, N-alkyliminodipropionic acids, N-hydroxyethyl-N-alkyl-amidopropylglycines, N-alkyltaurines, N-alkylsarcosines, 2-alkylaminopropionic acids, and alkylaminoacetic acids, each having about 8 to 24 C atoms in the alkyl group. Particularly preferred amphoteric surfactants are N-coco-alkylaminopropionate, coco-acylaminoethylaminopropionate, and C₁₂-C₁₈-acyl sarcosine.

It has also proven to be advantageous if the coloring and lightening agents according to the invention contain further, nonionogenic surface-active substances. Nonionic surfactants contain as a hydrophilic group, for example, a polyol group, a polyalkylene glycol ether group, or a combination of polyol- and polyglycol ether group. Such compounds include, for example
addition products of 1 to 50 mol of ethylene oxide and/or 0 to 5 mol of propylene oxide onto linear and branched fatty alcohols having 8 to 30 carbon atoms, such as for example lauryl, myristyl, cetyl, but also stearyl, isostearyl and oleyl alcohol, onto fatty acids having 8 to 30 carbon atoms and onto alkylphenols having 8 to 15 carbon atoms in the alkyl group,
addition products end-capped with a methyl- or C₂-C₆ alkyl functional group of 1 to 50 mol ethylene oxide alkyl and/or 0 to 5 mol propylene oxide to linear and branched fatty alcohols with 8 to 30 carbon atoms, to fatty acids with 8 to 30 carbon atoms, and to alkylphenols with 8 to 15 carbon atoms in the alkyl group, such as those available type under the trade names Dehydol® LS, Dehydol® LT (Cognis),
polyglycerol esters and alkoxylated polyglycerol esters, such as for example poly(3)glycerol diisostearate (commercial product: @LameformTGI (Henkel)) and poly(2)glycerol polyhydroxystearate (commercial product: Dehymuls®PGPH (Henkel)).
polyol fatty acid esters, such as the commercial product Hydagen® HSP (Cognis) or Sovermol-types (Cognis),
more highly alkoxylated, preferably propoxylated and in particular ethoxylated mono-, di- and triglycerides, such as glycerol monolaurate+20 ethylene oxide and glycerol monostearate+20 ethylene oxide,
amine oxides,
hydroxy mixed ethers,
sorbitan fatty acid esters and addition products of ethylene oxide to sorbitan fatty acid esters, such as for example polysorbates and sorbitol monolaurate+20 mol ethylene oxide (EO),
sugar fatty acid esters and addition products of ethylene oxide to sugar fatty acid esters,
addition products of ethylene oxide to fatty acid alkanolamides and fatty amines,
fatty acid-N-alkyl glucamides,
alkylphenols and alkylphenol alkoxylates having 6 to 21, in particular 6 to 15, carbon atoms in the alkyl chain and 1 to 30 ethylene oxide and/or propylene oxide units. Preferred representatives of this class include nonylphenol+9 EO and octylphenol+8 EO;
alkyl polyglycosides corresponding to the general formula RO—(Z)ₓ, wherein R denotes an alkyl, Z denotes a sugar, and x denotes the number of sugar units. The alkylpolyglycosides that can be used according to the invention can only contain a specific alkyl functional group R. According to the invention, however, these compounds are produced starting from natural fats and oils or mineral oils. In this case, R mixtures as alkyl radicals corresponding to the starting compounds or corresponding to the respective reprocessing of these compounds are present as alkyl functional groups.

Particularly suitable nonionic surfactants are C₈C₂₂-alkyl monoglycosides and -alkyl oligoglycosides and their ethoxylated analogs. In particular, non-ethoxylated compounds have proven to be particularly suitable.

Particularly preferred are those alkyl polyglycosides of the formula RO—(Z)ₓ where R
substantially consists of C₈ and C₁₀ alkyl groups,
substantially consists of C₁₂ and C₁₄ alkyl groups,
substantially consists of C₈ to C₁₆ alkyl groups or
substantially consists of C₁₂-to C₁₆ alky groups or
substantially consists of C₁₆ to C₁₈ alkyl groups.

These compounds are characterized in that any mono- or oligosaccharides can be used as sugar building block Z. Usually, sugars with 5 or 6 carbon atoms as well as the corresponding oligosaccharides are used. Such sugars are, for example, glucose, fructose, galactose, arabinose, ribose, xylose, lyxose, allose, altrose, mannose, gulose, idose, talose, and sucrose. Preferred sugar building blocks are glucose, fructose, galactose, arabinose and sucrose; glucose is particularly preferred.

The alkylpolyglycosides which can be used according to the invention contain on average 1.1 to 5 sugar units. Alkylpolyglycosides having x values of 1.1 to 2.0 are preferred. Very particular preference is given to alkylglycosides in which x is 1.1 to 1.8.

The alkoxylated homologs of said alkylpolyglycosides can also be used according to the invention. These homologs can contain on average up to 10 ethylene oxide and/or propylene oxide units per alkyl glycoside unit.

Addition products of alkylene oxide to saturated linear fatty alcohols and fatty acids containing from 2 to 30 mol ethylene oxide per mol of fatty alcohol or acid, have proved to be suitable as further preferred non-ionic surfactants. Preparations with excellent properties are likewise obtained if they contain fatty acid esters of ethoxylated glycerol as the nonionic surfactants.

Particularly preferred non-ionogenic surface-active substances are, because of the simple processability, substances which are commercially available in pure form as solids or liquids. In this context, the definition for purity does not refer to chemically pure compounds. Rather, in particular if the products have a natural base, mixtures of different homologues can be used, for example with different alkyl chain lengths, as obtained in products based on natural fats and oils. Mixtures of different degrees of alkoxylation are usually also present in alkoxylated products. In this context, the term purity refers instead to the fact that the selected substances should preferably be free of solvents, controls and other accompanying substances.

Products with a “normal” homolog distribution as well as those with a narrow homolog distribution may be used as surfactants which are addition products of ethylene and/or propylene oxide to fatty alcohols or derivatives of these addition products. “Normal” homolog distribution is understood to meanmixtures of homologs which are obtained during the conversion of fatty alcohol and alkylene oxide using alkali metals, alkali metal hydroxides or alkali metal alcoholates as catalysts. Narrowed homolog distributions, on the other hand, are obtained if, for example, hydrotalcite, alkaline earth metal salts of ether carboxylic acids, alkaline earth metal oxides, hydroxiides or alcoholoates are used as catalysts. The use of products with a narrow homolog distribution range may be preferred.

The anionic, nonionic, zwitterionic or amphoteric surfactants are used in amounts of 0.1 to 45 wt. %, preferably 1 to 30 wt. % and very particularly preferably 1 to 15 wt. %, based on the total amount of the ready-to-use agent.

Also preferred according to the invention are cationic surfactants of the quaternary ammonium compound, esterquat and amidoamine type. Preferred quaternary ammonium compounds are ammonium halides, in particular chlorides and bromides, such as alkyl trimethyl ammonium chlorides, dialkyldimethyl ammonium chlorides and trialkyl methyl ammonium chlorides, e.g., cetyltrimethylammonium chloride, stearyltrimethylammonium chloride, distearyldimethyl ammonium chloride, lauryldimethyl ammonium chloride, lauryldimethylbenzyl ammonium chloride, and tricetylmethyl ammonium chloride, and the imidazolium compounds known under the INCI names Quaternium-27 and Quaternium-83. The long alkyl chains of the above-mentioned surfactants preferably have 10 to 18 carbon atoms. The quaternized protein hydrolysates represent other cationic surfactants that can be used according to the invention.

The alkylamidoamines are usually prepared by amidation of natural or synthetic fatty acids and fatty acid fractions with dialkylaminoamines and are characterized by their good biodegradability alongside a good conditioning effect. One compound from this substance group which is particularly suitable according to the invention is the stearamidopropyl dimethylamine available commercially under the name Tegoamid® S 18.

Quaternary ester compounds, referred to as “esterquats”, are likewise very readily biodegradable. Esterquats are known substances that contain both at least one ester function and at least one quaternary ammonium group as structural element. Preferred esterquats are quaternized ester salts of fatty acids with tritethanoamine, quanterized ester salts of fatty acids with diethanolalkylamines and quaternized ester salts of fatty acids with 1,2-dihydroxypropyldialkylamines. Such products are sold for example under the trademarks Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such esterquats.

The cationic surfactants are contained in the agents used according to the invention preferably in amounts of 0.05 to 10 wt. %, based on the total agent. Particular preference is given to amounts of 0.1 to 5 wt. %.

In one preferred embodiment, preference may be given to nonionic, zwitterionic and/or amphoteric surfactants and mixtures thereof.

In a further preferred embodiment, the effect of the active ingredient according to the invention can be enhanced by emulsifiers. Such emulsifiers are for example
addition products of 4 to 30 mol of ethylene oxide and/or 0 to 5 mol of propylene oxide to linear fatty alcohols having 8 to 22 C atoms, to fatty acids having 12 to 22 C atoms, and to alkylphenols having 8 to 15 C atoms in the alkyl group,
C₁₂-C₂₂ fatty acid monoesters and diesters of addition products of 1 to 30 mol of ethylene oxide of polyols having 3 to 6 carbon atoms, in particular onto glycerol,
ethylene oxide and polyglycerol addition products of methyl glucoside fatty acid esters, fatty acid alkanolamides, and fatty acid glucamides,
C₈-C₂₂ alkyl mono- and oligoglycosides and the ethoxylated analogs thereof, preference being given to degrees of oligomerization of 1.1 to 5, in particular 1.2 to 2.0, and glucose as the sugar component,
mixtures of alkyl (oligo) glucosides and fatty alcohols, for example, the commercially available product Montanov® 68, addition products of 5 to 60 mol ethylene oxide to castor oil and hydrogenated castor oil,
partial esters of polyols having 3 to 6 carbon atoms with saturated fatty acids having 8 to 22 carbon atoms, sterols, sterols being understood to mean a group of steroids which carry a hydroxyl group on carbon atom 3 of the steroid backbone and are isolated both from animal tissue (zoo sterols) and plant fats (phytosterols). Examples of zoosterols include cholesterol and lanosterol. Examples of suitable phytosterols include ergosterol, stigmasterol, and sitosterol. There are also sterols that are isolated from fungi and yeasts (so-called mycosterols).
phospholipids, especially glucose phospholipids, which are obtained for example as lecithins or phosphatidylcholines from for example egg yolk or plant seeds (for example soya beans),
fatty acid esters of sugars and sugar alcohols, such as sorbitol,
polyglycerols and polyglycerol derivatives, such as for example polyglycerol poly-12-hydroxystearate (commercial product: Dehymuls® PGPH)
linear and branched fatty acids having 8 to 30 carbon atoms, and the Na, K, ammonium, Ca, Mg and Zn salts thereof.

The agent according to the invention contain the emulsifiers preferably in amounts of 0.1 to 25 wt. % in particular 0.5 to 15 wt. % based on the total amount of the ready-to-use agent.

According to the invention, particular preference may be given to nonionogenic emulsifiers and surfactants having an HLB value of 10-15. Among these emulsifier types, very particular preference may be given to those emulsifiers which contain no ethylene oxide and/or propylene oxide in the molecule.

Further work has shown that the oxidative formation of intense colorations from the components (a) and (b) functions in particular in the cosmetic carrier formulations the fat component content of which is not excessively high. A strong color application was then observed in particular if, based on the total weight of the agent, the total amount of the fat components contained in the agent was below 25 wt. %, preferably below 20 wt. %, more preferably below 15 wt. %, and very particularly preferably below 13 wt. %.

In the context of a further preferred embodiment, an agent according to the invention is characterized in that, based on the total weight of the agent, the total content of the fatty components contained in the agent is below 25 wt. %, preferably below 20 wt. %, further preferably below 15 wt. %, and very particularly preferably below 13 wt. %.

Fatty components within the context of the invention are understood to be organic compounds with a solubility in water of less than 1 wt. %, and preferably less than 0.1 wt. % at room temperature (22° C.) and atmospheric pressure (760 mmHg).

Under the definition of fat components fall explicitly only uncharged (i.e., non-ionic) compounds. Fat constituents have at least one saturated or unsaturated alkyl group having at least 8 C atoms. The molecular weight of the fat component is at most 5,000 g/mol, preferably at most 2,500 g/mol, and particularly preferably at most 1,000 g/mol. The fat components are either polyoxyalkylated or polyglycerylated compounds.

In this context, fatty components are understood to be components from the group of C₁₂-C₃₀ fatty alcohols, C₁₂C₃₀ fatty acid triglycerides and/or hydrocarbons. Within the meaning of the present invention only non-ionic substances are explicitly considered as fatty components. Charged compounds, such as fatty acids and salts thereof, are not understood to be fatty components.

C₁₂-C₃₀ fatty alcohols can be saturated, mono- or polyunsaturated, linear or branched fatty alcohols having 12 to 30 C atoms.

Examples of preferred linear saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Linear unsaturated fatty alcohols are, for example, (9Z)-octadec-9-en-1-ol (oleyl alcohol), (9E)-octadec-9-en-1-ol (elaidyl alcohol), (9,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

For a C₁₂-C₃₀ fatty acid triglyceride, in the context of the present invention, the triesters of trivalent alcohol glycerol are understood with three equivalents of fatty acid. Both structurally similar and different fatty acids may be involved in the ester formation within a triglyceride molecule.

According to the invention, fatty acids are to be understood as saturated or unsaturated, unbranched or branched, unsubstituted or substituted C₁₂-C₃₀ carboxylic acids. Unsaturated fatty acids may be mono-unsaturated or poly-unsaturated. With an unsaturated fatty acid, the C═C double bond(s) thereof may have the cis or trans configuration.

By way of example, esters originating from glycerol having a fatty acid can be named as fatty acid triglycerides, wherein the fatty acid is selected from the group consisting of dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The fatty acid triglycerides may also be of natural origin. The fatty acid triglycerides or mixtures thereof, for example corresponding natural fatty acid triglycerides, occurring in soybean oil, groundnut oil, olive oil, sunflower oil, macadamia nut oil, moringa oil, apricot kernel oil, marula oil and/or optionally hydrogenated castor oil.

Hydrocarbons are compounds having 8 to 80 C atoms composed exclusively of carbon and hydrogen atoms. Especially preferred in this context are aliphatic hydrocarbons, such as mineral oils, liquid paraffin oils (e.g., paraffinum liquidum or paraffinum perliquidum), isoparaffin oils, semisolid paraffin oils, paraffin waxes, solid paraffin (paraffinum solidum), Vaseline, and polydecene.

In this respect, liquid paraffin oils (Paraffinum Liquidum and Paraffinum Perliquidum) have proven to be particularly suitable. The hydrocarbon is very particularly preferably Paraffinum Liquidum, also referred to as white oil. Paraffinum Liquidum is a mixture of purified, saturated, aliphatic hydrocarbons, consisting mainly of hydrocarbon chains having a C-chain distribution of from 25 to 35 C atoms.

The dyes according to the invention can preferably contain further auxiliary substances and additives. For instance, it has proven to be preferred according to the invention if the agents contain at least one thickening agent. In principle, there are no restrictions with regard to these thickeners. Both organic and purely inorganic thickeners may be used.

According to a first preferred embodiment, the thickening agent is an anionic, synthetic polymer. Preferred anionic groups are the carboxylate group and the sulfonate group.

The examples of anionic monomers, which the polymeric anionic thickener may consist of, are acrylic acid, methacrylic acid, crotonic acid, itaconic acid, maleic anhydride and 2-acrylamido-2-methylpropanesulfonic acid. Here, the acidic groups may be present wholly or partly as sodium-, potassium-, ammonium-, mono- or triethanolammonium salt. Preferred monomers are maleic anhydride and especially 2-acrylamido-2-methyl-propane-sulfonic acid and acrylic acid.

Preferred anionic homopolymers are uncrosslinked and crosslinked polyacrylic acids. Here, allyl ethers of pentaerythritol, sucrose and propylene may be preferred crosslinking agents. Such compounds are commercially available, for example, under the trade name Carbopol®. Also preferred is the homopolymer of 2-acrylamido-2-methyl propane sulfonic acid, which is commercially available, for example, under the name Rheothik®11-80.

Within this first embodiment, it may further be preferred to use copolymers of at least one anionic monomer and at least one nonionic monomer. With regard to the anionic monomers, reference is made to the substances listed above. Preferred non-ionic monomers are acrylamide, methacrylamide, acrylic acid esters, methacrylic acid esters, itaconic acid monoesters and diesters, vinyl pyrrolidinone, vinyl ethers, and vinyl esters.

The anionic acrylic acid and/or methacrylic acid polymers or copolymers are contained in the agents according to the invention preferably in an amount of 0.1 to 10% by weight, particularly preferably 1 to 5% by weight, in each case based on the weight of the agent.

Preferred anionic copolymers are, for example, copolymers of acrylic acid, methacrylic acid or their C₁-C₆-alkyl esters, such as are marketed under the INCI name acrylate copolymers. One preferred commercial product is Aculyn® 33 from Rohm & Haas, for example. However, copolymers of acrylic acid, methacrylic acid or their C₁-C₆ alkyl esters and the esters of an ethylenically unsaturated acid and an alkoxylated fatty alcohol are further preferred. Suitable ethylenically unsaturated acids are in particular acrylic acid, methacrylic acid and itaconic acid; suitable alkoxylated fatty alcohols are in particular Steareth-20 or Ceteth-20. Such copolymers are marketed by Rohm & Haas under the trade name Aculyn®22 and by National Starch under the trade names Structure@2001 and Structure®3001.

Further preferred anionic copolymers are acrylic acid-acrylamide-copolymers as well as in particular polyacrylamide copolymers with monomers containing a sulfonic acid group. A particularly preferred anionic copolymer consists of 70 to 55 mol % of acrylamide and 30 to 45 mol % of 2-acrylamido-2-methylpropane sulfonic acid, wherein the sulfonic acid group is wholly or partially present as sodium-, potassium-, ammonium-, mono- or triethanolammonium salt. This copolymer can also be cross-linked, wherein preferably polyolefinically unsaturated compounds such as tetraallyloxythane, allylsucrose, allylpentaerythritol and methylene-bisacrylamide are used as crosslinking agents. Such a polymer is contained in the commercial products Sepigel®305 and Simulgel® 600 from the company SEPPIC. The use of these compounds, which contain a hydrocarbon mixture (C₁₃-C₁₄-isoparaffin or isohexadecane) and a non-ionic emulsifier (Laureth-7 or Polysorbate-80) in addition to the polymer components, has proved to be particularly advantageous in the context of the teaching of the invention.

Also, polymers of maleic acid anhydride and methyl vinyl ether, in particular those with crosslinks, are preferred thickeners. The maleic acid methyl vinyl ether copolymer cross-linked with 1,9-decadiene is available under the name Stabileze®QM.

According to another embodiment, the thickener is a cationic synthetic polymer. Preferred cationic groups are quaternary ammonium groups. In particular, those polymers, in which the quaternary ammonium group is bonded to a polymer backbone built-up of acrylic acid, methacrylic acid or derivatives thereof via a C₁-C₄ hydrocarbon group, have been found to be particularly suitable.

Homopolymers of general formula (HP-1),

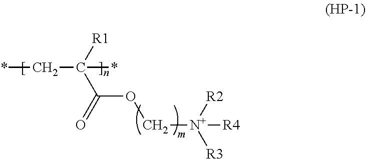

in which R1═—H or —CH₃, R2, R3 and R4 independently of one another are selected from C₁-C₄ alkyl, alkenyl or hydroxyalkyl groups, m=1, 2, 3 or 4, n is a natural number and X-is a physiologically acceptable organic or inorganic anion, as well as copolymers consisting substantially of the monomer units shown in formula (HP-1) and nonionogenic monomer units, are particularly preferred cationic polymeric gel formers. In the context of these polymers, those are preferred according to the invention, for which at least one of the following conditions applies:
R1 represents a methyl group
R2, R3 and R4 are methyl groups,
m has the value 2,

As a physiologically acceptable counter ion X—, for example, halide ions, sulfate ions, phosphate ions, methosulfate ions as well as organic ions such as lactate-, citrate-, tartrate- and acetate ions are considered. Halide ions are preferred, in particular chloride.

A particularly suitable homopolymer is the poly(methacryloxyethyltrimethylammonium) chloride (cross-linked, if desired) having the INCI name Polyquaternium-37. The crosslinking can be carried out, if desired, with the help of olefinically poly-unsaturated compounds, for example, divinylbenzene, tetraallyloxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably used in the form of a non-aqueous polymer dispersion, which should not have a polymer content of less than 30 wt.-%. Such polymer dispersions are available under the names Salcare®SC 95 (approx. 50% polymer content, further component: mineral oil (INCI name: mineral oil) and tridecyl-polyoxy-propylene-polyoxyethylene ether (INCI name: PPG-1-trideceth-6) and Salcare® SC 96 (approx. 50% polymer content, further components: mixture of diesters of propylene glycol with a mixture of caprylic and capric acid (INCI name: propylene glycol dicaprylate/dicaprate) and tridecyl-polyoxy-propylene-polyoxyethylene ether (INCI name: PPG-1-trideceth-6).

Copolymers comprising monomer units according to formula (HP-1) contain as nonionogenic monomer units preferably acrylamide, methacrylamide, acrylic acid C₁-C₄ alkyl ester and methacrylic acid C₁-C₄ alkyl ester. Among these, non-ionic monomers, acrylamide is particularly preferred. These copolymers can also be cross-linked like the homopolymers described above. A preferred copolymer according to the invention is a crosslinked acrylamide methacroyl oxyethyl trimethyl ammonium chloride copolymer. Such copolymers, in which the monomers are present in a weight ratio of about 20:80, are commercially available as approx. 50% non-aqueous poymer dispersion under the name Salcare® SC 92.

In another preferred embodiment, naturally occurring thickeners are used. Preferred thickeners of this embodiment are, for example, nonionic guar gums. According to the invention, both modified and unmodified guar gums can be used. Unmodified guar gums are marketed, for example, under the trade name Jaguar® C from Rhone Poulenc. Modified guar gums which are preferred according to the invention contain C₁-C₆ hydroxyalkyl groups. The groups are preferably hydroxymethyl, hydroxyethyl, hydroxypropyl and hydroxybutyl. Such modified guar gums are well known in the art and can be prepared, for example, by reacting the guar gums with alkylene oxides. The degree of hydroxyalkylation, which corresponds to the number of used alkylene oxide molecules in proportion to the number of the free hydroxyl groups of guar gum, is preferably between 0.4 to 1.2. Such modified guar gums are commercially available under the trade names Jaguar® HP8, Jaguar® HP60, Jaguar® HP120, Jaguar® DC 293 and Jaguar® HP105 from Rhone Poulenc.

Further suitable natural thickening agents are also already known from the prior art.

According to this embodiment, biosaccharide gums of microbial origin, such as scleroglucan gums or xanthan gums, gums from plant exudates, such as gum arabic, ghatti gum, karaya gum, tragacanth gum, carrageenan gum, agar-agar, locust bean gum, pectins, alginates, starch fractions and derivatives, such as amylose, amylopectin and dextrins, cellulose derivatives, such as methylcellulose, carboxyalkylcelluloses and hydroxyalkylcelluloses, are preferred.

Preferred hydroxyalkylcelluloses are, in particular, the hydroxyethylcelluloses, which are marketed under the names Cellosize® from Amerchol and Natrosol® from Hercules. Suitable carboxyalkyl celluloses are in particular the carboxymethylcelluloses as marketed under the names Blanose® from Aqualon, Aquasorb® and Ambergum® from Hercules and Cellgon® from Montello.

Preference is also given to starch and derivatives thereof. Starch is a storage material of plants, which occurs mainly in tubers and roots, in grain seeds, and in fruits, and can be obtained from a variety of plants in high yield. The polysaccharide, which is insoluble in cold water and forms a colloidal solution in boiling water, for example, can be obtained from potatoes, manioc, sweet potatoes, maranta, corn, grains, rice, legumes, such as peas and beans, bananas or the marrow of certain types of palm (for example, the sago palm). According to the invention, natural, plant-derived starches and/or chemically or physically modified starches can be used. Modification can be achieved, for example, by introducing different functional groups on one or more of the hydroxyl groups of the starch. These are usually esters, ethers or amides of starch having optionally substituted C1-C40 radicals. Particularly advantageous is an etherified corn starch with a 2-hydroxypropyl group, as is marketed, for example, by National Starch under the trade name Amaze®

However, nonionic, fully synthetic polymers, such as for example polyvinyl alcohol or polyvinylpyrrolidone, can also be used as thickening agents according to the invention. Preferred nonionic, fully synthetic polymers are marketed for example by BASF under the trade name Luviskol®. Such nonionic polymers also allow, in addition to their excellent thickening properties, a significant improvement in the sensory feeling of the resulting preparations.

As inorganic thickeners, phyllosilicates (polymeric, crystalline sodium disilicates) have proven to be particularly suitable in the context of the present invention. In particular tone, in particular, magnesium aluminum silicates, such as bentonite, particularly smectites, such as montmorillonite or hectorite, which may also be optionally suitably modified, and synthetic phyllosilicates, such as the magnesium phyllosilicates marketed by the company Sud Chemie under the trade name Optigel®, are preferred.

To further increase the performance of the oxidative dyes, at least one optionally hydrated SiO₂ compound is additionally preferably added. It may be preferred according to the invention to use the optionally hydrated SiO₂ compounds in amounts of 0.05 wt. % to 15 wt. %, particularly preferably in amounts of 0.15 wt. % to 10 wt. % and very particularly preferably in amounts of 0.2 wt. % to 5 wt. %, in each case based on the composition. The specified amounts in each case reflect here the content of the SiO₂ compounds (without the water content thereof) in the agents.

With regard to the optionally hydrated SiO₂ compounds, the present invention is in principle subject to no limitations. Preference is given to silicic acids, oligomers thereof and polymers thereof, and also salts thereof. Preferred salts are the alkali metal salts, in particular the potassium and sodium salts. The sodium salts are very particularly preferred.

The optionally hydrated SiO₂ compounds may be present in different forms. According to the invention, preference is given to using the SiO₂ compounds in the form of silica gels, or particularly preferably as water glass. These SiO₂ compounds may sometimes be present in aqueous solution.

Very particularly preferred according to the invention are water glasses, which are formed from a silicate of formula (SiO₂)ₙ(Na₂O)ₘ(K₂O)ₚ, where n represents a positive rational number, and m and p, independently of one another, represent a positive rational number or 0, with the proviso that at least one of the parameters m or p be different from 0, and the ratio between n and the sum of m and p be between 1:4 and 4:1, are likewise preferred. Preference is given to metasilicates in which the ratio between n and the sum of m and p is 1:2 or below.

Besides the components described by the empirical formula, the water glasses may also contain further additives in small amounts, such as for example phosphates or magnesium salts.

Water glasses which are particularly preferred according to the invention are marketed inter alia by Henkel under the names Ferrosil® 119, Natronwasserglas 40/42, Portil® A, Portil® AW and Portil® W and by Akzo under the name Britesil® C20.

The oxidation dyes are preferably packaged as flowable preparations.

The agents according to the invention may also contain further active substances, auxiliaries and additives, such as for example
nonionic polymers, such vinylpyrrolidinone/vinylacrylate copolymers, polyvinylpyrrolidinone, vinylpyrrolidone/vinyl acetate copolymers, polyethylene glycols and polysiloxanes;
silicones, such as volatile or non-volatile, straight-chain, branched or cyclic, crosslinked or non-crosslinked polyalkylsiloxanes (such as dimethicones or cyclomethicones), polyarylsiloxanes and/or polyalkylarylsiloxanes, in particular polysiloxanes having organofunctional groups, such as substituted or unsubstituted amines (amodimethicones), carboxyl, alkoxy and/or hydroxyl groups (dimethicone copolyols), linear polysiloxane (A)/polyoxyalkylene (B) block copolymers, grafted silicone polymers having a non-silicone-containing organic backbone or having a polysiloxane backbone, such as for example the commercial product Abil B 8832 from Degussa, which is marketed under the INCI name Bis-PEG/PPG-20/20 Dimethicone, or mixtures thereof;
cationic polymers, such as quaternized cellulose ethers, polysiloxanes having quaternary groups, dimethyldiallylammonium chloride polymers, acrylicamide dimethyldiallylammonium chloride copolymers, with diethyl sulfate quaternary dimethylaminoethylmethacrylate vinyl pyrrolidinone copolymers, vinylpyrrolidinone imidazolinium methochloride copolymers, and quaternized polyvinyl alcohol,
zwitterionic and amphoteric polymers, such as, for example, acrylic-amidopro-pyl-tri-methyl ammonium chloride/acrylate copolymers and Octyl-acrylic amide/-Me-thyl-methacrylic/tert-butylaminoethyl methacrylate-Co-late/2-Hydroxypro-pyl-methacrylate-Co-polymeric, Diallyl-dimethylammonium chloride/acrylate copolymers, t-butylaminoethyl methacrylate/N-(1,1,3,3-tetramethylbutyl) acrylic amide/acrylate (/methacrylate) copolymers,
anionic polymers, such as, for example, polyacrylic acids, crosslinked polyacrylic acids, vinyl acetate/crotonic acid copolymers, vinylpyroirinon/vinyl acrylate copolymers, vinyl acetate/butyl maleate/isoobornylacrylate copolymers, methyl vinyl ether/maleic acid anhydride copolymers, and acrylic acid/ethylacrylate/N-t-butyl acrylamide terpolymers,
further thickening agents such as agar, Guar gum, alginates, xanthan gum, gum arabicum, karaya gum, locust bean gum, linseed-gums, dextranes, cellulose derivatives, e.g., methyl cellulose, hydroxyalkylcellulose and carboxymethylcellulose, starch fractions and derivatives, such as amylose, amylopectin, and dextrines, clays. such as bentonite or fully synthetic hydroglycol, such as, for example, polyvinylalcohol,
structurants, such as glucose, maleic acid and lactic acid,
hair-conditioning compounds, such as phospholipids, for example soya lecithin, egg lecithin and cephalins as well as silicone oils,
perfume oils, dimethyl isosorbide and cyclodextrins,
solvents and solubilizers, such as ethanol, isopropanol, ethylene glycol, propylene glycol, glycerol, and diethylene glycol,
fiber-structure-improving active ingredients, in particular mono-, di-, and oligosaccharides, such as, for example, glucose, galactose, fructose, fruit sugar, and lactose.
quaternized amines such as methyl-1-alkylamidoethyl-2-alkylimidazolinium methosulfate
defoaming agents, such as silicones,
dyes for coloring the agent,
anti-dandruff active substances such as piroctone olamine, zinc omadine and climbazole,
amino acids and oligopeptides, in particular arginine and/or serine,
protein hydrolysates of animal and/or plant origin, such as for example elastin, collagen, keratin, silk and lactoprotein protein hydrolysates, or almond, rice, pea, potato and wheat protein hydrolysates, as well as those in the form of their fatty acid condensation products or optionally anionically or cationically modified derivatives thereof;
vegetable oils, for example macadamia nut oil, kukui nut oil, palm oil, amaranth seed oil, peach kernel oil, avocado oil, olive oil, coconut oil, rapeseed oil, sesame oil, jojoba oil, soybean oil, peanut oil, evening primrose oil and tea tree oil
light stabilizers, in particular derivatized benzophenones, cinnamic acid derivatives and triazines,
substances for adjusting the pH, such as for example conventional acids, in particular edible acids and bases;
active substances, such as panthenol, pantothenic acid, pantolactone, allantoin, pyrrolidinone carboxylic acids and the salts thereof, and bisabolol,
polyphenols, in particular hydroxycinnamic acids, 6,7-dihydroxycoumarin, hydroxybenzoic acids, catechins, tannins, leucoanthocyanidins, anthocyanidins, flavanones, flavones, and flavonols;
ceramides, preferably the sphingolipids such as ceramide I, ceramide II, ceramide 1, ceramide 2, ceramide 3, ceramide 5 and ceramide 6, or pseudoceramides, such as in particular N—(C8-C22-acyl)-(C8-C22-acyl)-hydroxyproline,
vitamins, provitamins and vitamin precursors, in particular those of groups A, B3, B5,B6, C, E, F and H,
plant extracts, such as for example the extracts of aloe vera, angelica, anise, apricot, benzoin, bergamot, birch, nettle, calamus, blackcurrant, costus, hibiscus, oak bark, elemi, tarragon, pine needles, galbanum, geranium, ginseng, grapefruit, guaiac wood, green tea, hamamelis, restharrow, hops, coltsfoot, ginger root, iris, jasmine, chamomile, cardamom, clover, burdock root, pine, kiwi fruit, coconut, coriander, caraway, mountain pine, lavender, lemon grass, lily, lime, linden blossom, lychee, mace, malva, almond, mango, lemon balm, melon, meristem, myrrh, neroli, olibanum, opoponax, orange, patchouli, petitgrain, stone pine, wild thyme, rooibos, rose, rosemary, horse chestnut, sandalwood, sage, horsetail, yarrow, celery, spruce, thyme, juniper, vine leaves, hawthorn, wheat, lady's-smock, ylang-ylang, cedar and lemon.
cholesterol,
consistency regulators, such as sugar esters, polyol esters or polyol alkyl ethers;,
fats and waxes, such as spermaceti, beeswax, montan wax and paraffins;,
fatty acid alkanolamides,
swelling and penetration substances, such as glycerol, propylene glycol monoethyl ether, carbonates, hydrogencarbonates, guanidines, ureas, and also primary, secondary and tertiary phosphates,
turbidity agents such as latex, styrene/PVP and styrene/acrylamide copolymers;
pearlescent agents, such as ethylene glycol mono- and distearate and PEG-3 distearate,
pigments,
stabilizing agents for hydrogen peroxide and other oxidizing agents,
propellants such as propane-butane mixtures, N₂O, dimethyl ether, CO₂ and air,
antioxidants.

The selection of these additional substances is made by the person skilled in the art according to the desired properties of the agents.

With regard to other optional components and the amounts of said components used, reference is explicitly made to relevant handbooks known to a person skilled in the art. Schrader, Grundlagen and Rezepturen der Kosmetika, 2nd edition, Hüthig Buch Verlag, Heidelberg, 1989.

The additional active ingredients and auxiliaries are used in the agents according to the invention preferably in quantities of from, in each case, 0.0001 to 10 wt.-%, in particular of from 0.0005 to 5 wt.-%, relative to the total weight of the application mixture.

### Multi-Component Packaging Unit (Kit-of-Parts)

The agent according to the invention are agents for oxidatively dyeing or dyeing and lightening hair. In the ready-to-use agent, the oxidation dye precursors react with the oxidizing agent to form the actual dyeing agents. The agents according to the invention are therefore usually packaged as multicomponent agents, usually as two-component products. In this case, the first component contains the oxidation dye precursors (a) and isatin (b), which is mixed shortly before application with a second component containing the oxidizing agent (e) and the complexing agent or the complexing agents (c). Usually, both components are mixed with one another in the range of from 1:3 to 3:1, preferably from 1:2 to 2:1. This mixture of the component containing color cream and optionally alkalizing agent (preparation A) and the component containing oxidizing agent (preparation B) is referred to as the application mixture or the ready-to-use agent.

A second subject of the present invention is a multi-component packaging unit (kit-of-parts) for oxidatively coloring keratin fibers, in particular human hair, comprising, packaged separately from one another,
a first container having a dye (F), which contains
(a) at least one developer-type oxidation dye precursor, and
(b) isatin and
a second container having an oxidizing agent composition (Ox), which contains
(c) at least one complexing agent from the group consisting of ethylenediamine disuccinic acid (EDDS), L-glutamic acid-N,N-diacetic acid (GLDA), N,N-bis(carboxymethyl)-L-alanine (MGDA), 1-hydroxyethane-1,1-diphosphonic acid (HEDP), ethylenediaminetetraacetic acid (EDTA), ethylenediamine tetramethylene phosphonic acid (EDTMP), diethylene triamine pentamethylene phosphonic acid (DTPMP), pyridine-2,6-dicarboxylic acid, and the physiologically acceptable salts thereof, and
(e) at least one oxidizing agent from the group consisting of hydrogen peroxide and its addition products of organic or inorganic compounds
the components (a), (b), (c) and (e) having already been disclosed in detail in the description of the first object of the invention.

Alternatively, the dye can also be referred to as color cream.

### Method for Oxidatively Dyeing Keratin Fibers

The oxidative dye according to the invention of the first subject matter of the invention or the multicomponent packaging unit, the separately packaged components of which are used for producing said ready-to-use dye, are outstandingly suitable for use in corresponding dyeing methods,

A further subject matter of the present invention is therefore a method for oxidatively dyeing keratinous fibers, in particular human hair, in which a means, as disclosed in detail in the description of the first subject matter of the invention, is applied to the keratinous fibers and rinsed out again after an exposure time.

While the fibers are being exposed to the agent, it can be advantageous to support the dyeing process by applying heat. Heat can be applied by an external heat source, such as e.g. hot air from a hot-air blower, and also, in particular in the case of dyeing the hair of a living subject, by the body temperature of the subject. In the latter possibility, conventionally the part to be dyed is covered with a cap. In particular, the temperature during the exposure time is between 10° C. and 45° C., in particular between 20° C. and 40° C. The dyeing agents according to the invention already give intense colorations at physiologically acceptable temperatures of less than 45° C. Therefore they are suitable particularly for coloring human hair.

What has been stated regarding the means according to the invention applies, mutatis mutandis, to additional preferred embodiments of the multicomponent packaging unit and method according to the invention.

What is claimed is:

1. An agent for oxidatively dyeing keratinous fibers, in particular human hair, containing, in a cosmetic carrier,
(a) at least one developer-type oxidation dye precursor,
(b) isatin, and
(c) at least one complexing agent from the group consisting of ethylenediamine disuccinic acid (EDDS), L-glutamic acid-N,N-diacetic acid (GLDA), N,N-bis(carboxymethyl)-L-alanine (MGDA), 1-hydroxyethane-1,1-diphosphonic acid (HEDP), ethylenediaminetetraacetic acid (EDTA), ethylenediamine tetramethylene phosphonic acid (EDTMP), diethylene triamine pentamethylene phosphonic acid (DTPMP), pyridine-2,6-dicarboxylic acid, and the physiologically acceptable salts thereof.

2. The agent according to claim 1, wherein the at least one developer-type oxidation dye precursor (a) is selected from the group consisting of p-toluylene diamine, 2-methoxymethyl-p-phenylenediamine, 2-(2-hydroxy-ethyl)-p-phenylenediamine, N,N-bis-(2-hydroxyethyl)-p-phenylenediamine, p-phenylenediamine, 4-amino-3-methylphenol, p-aminophenol, 4,5-diamino-1-(2-hydroxyethyl) pyrazole, and the physiologically acceptable salts thereof.

3. The agent according to claim 1, wherein, based on the total weight of the agent, the at least one developer-type oxidation dye precursor (a) is present in a total amount of 0.001 to 10.0 wt. %.

4. The agent according to claim 1, wherein, based on the total weight of the agent, the at least one developer-type oxidation dye precursor (a) is present in a total amount of 0.1 to 5.0 wt. %.

5. The agent according to claim 1, wherein, based on the total weight of the agent, isatin (b) is present in a total amount of 0.001 to 10 wt. %.

6. The agent according to claim 1, wherein, based on the total weight of the agent, isatin (b) is present in a total amount of 0.1 to 3.5 wt. %.

7. The agent according to claim 1, wherein the weight ration of all all developers of group (a) contained in the agent to the isatin (b) contained in the agent has a value of 2:1 to 1:2.

8. The agent according to claim 1, wherein the weight ration of all all developers of group (a) contained in the agent to the isatin (b) contained in the agent has a value of 1.8:1 to 1.2:1.

9. The agent according to claim 1, further comprising at least one coupler-type oxidation dye precursor (d) selected from the group consisting of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2 (2,4-diaminophenoxy) ethanol, 1,3-bis(2,4-diaminophenoxy) propane, 1-methoxy-2-amino-4-(2-hydroxyethylamino)benzene, 1,3-bis(2,4-diaminophenyl) propane, 2,6-bis(2′-hydroxyethyl)-1-methylbenzene, 1-amino-3-bis(2-hydroxyethyl)aminobenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

10. The agent according to claim 9, wherein, based on the total weight of the agent, the at least one coupler-type oxidation dye precursor is present in a total amount of 0.001 to 10 wt. %.

11. The agent according to claim 9, wherein, based on the total weight of the agent, the at least one coupler-type oxidation dye precursor is present in a total amount of 0.1 to 3.5 wt. %.

12. The agent according to claim 1, wherein, relative to the total weight of the agent, the total amount of oxidation dye precursor of the resorcinol-type contained in the agent is below below 0.1 wt %.

13. The agent according to claim 1, further comprising at least one oxidizing agent(s) selected from the group consisting of hydrogen peroxide and its addition products of organic or inorganic compounds.

14. The agent according to claim 1, wherein the at least one complexing agent (c) is selected from the group consisting of ethylenediamine disuccinic acid (EDDS), L-glutamic acid-N,N-diacetic acid (GLDA), N,N-bis(carboxymethyl)-L-alanine (MGDA) and physiologically acceptable salts thereof.

15. The agent according to claim 1, wherein, based on the total weight of the agent, the one or more complexing agents (c) are present in a total amount of 0.05 to 10.0 wt. %.

16. The agent according to claim 1, wherein, based on the total weight of the agent, the one or more complexing agents (c) are present in a total amount of 0.15 to 2.5 wt. %.

17. The agent according to claim 1, further comprising at least one alkalizing agent selected from the group consisting of ammonia, monoethanolamine, 2-amino-2-methylpropanol, arginine, lysine, ornithine, and histidine.

18. The agent according to claim 1, wherein, based on the total weight of the agent, the total content of the fatty components contained in the agent is below 25 wt %.

19. A multi-component packaging unit (kit-of-parts) for oxidatively dyeing keratinous fibers, in particular human hair, comprising, packaged separately from one another,
a first container having a dye (F), which contains
(a) at least one developer-type oxidation dye precursor, and
(b) isatin and
a second container having an oxidizing agent composition (Ox), which contains
(c) at least one complexing agent selected from the group consisting of ethylenediamine disuccinic acid (EDDS), L-glutamic acid-N,N-diacetic acid (GLDA), N,N-bis(carboxymethyl)-L-alanine (MGDA), 1-hydroxyethane-1,1-diphosphonic acid (HEDP), ethylenediaminetetraacetic acid (EDTA), ethylenediamine tetramethylene phosphonic acid (EDTMP), diethylene triamine pentamethylene phosphonic acid (DTPMP), pyridine-2,6-dicarboxylic acid, and the physiologically acceptable salts thereof, and
(d) at least one oxidizing agent from the group consisting of hydrogen peroxide and its addition products of organic or inorganic compounds.

20. A method for oxidatively dyeing keratinous fibers, in particular human hair, wherein an agent according to claim 1 is applied to the keratinous fibers and rinsed out again after an exposure time.
